(19) United States
(12) Patent Application Publication
(10) Pub. No.: US 2010/0286422 A1
(43) Pub. Date: Nov. 11, 2010

(54) ENZYME RECOVERY SORBENT, ENZYME RECOVERY UNIT, LIGNOCELLULOSIC BIOREFINERY, PROCESS FOR RECYCLING ENZYMES, AND RENEWABLE MATERIAL

(75) Inventor: Jacob Borden, Chicago, IL (US)

Correspondence Address: CAROL WILSON;BP AMERICA INC., MAIL CODE 5 EAST, 4101 WINFIELD ROAD, WARRENVILLE, IL, 60555, US

(73) Assignee: BP Corporation North America Inc., Warrenville, IL (US)

(21) Appl. No.: 12/437,891

(22) Filed: May 8, 2009

(51) Int. Cl. C07C 57/00; C12M 1/00; C12M 1/12; C12N 9/00; C07C 31/08; C07C 59/08; C07C 53/08; C07C 31/12; C07C 53/122

(52) U.S. Cl. 554/227; 435/283.1; 435/297.1; 435/183; 568/840; 554/1; 562/579; 562/512

(57) ABSTRACT

This invention relates to an enzyme recovery sorbent, an enzyme recovery unit, a lignocellulosic biorefinery, a process for recycling enzymes, and a renewable material. The invention includes a lytic enzyme recovery sorbent suitable for use in production of renewable materials. The sorbent includes a substrate, and an enzyme binding material dispersed with respect to the substrate.

### BACKGROUND

1. Technical Field

This invention relates to an enzyme recovery sorbent, an enzyme recovery unit, a lignocellulosic biorefinery, a process for recycling enzymes, and a renewable material.

2. Discussion of Related Art

Tightening oil supply and escalating energy prices along with environmental concerns over nonrenewable resources have prompted significant interest and research into renewable materials and/or biofuels. Efforts to reduce carbon emissions and greenhouse gases are also driving investment into renewable materials and/or biofuels.

One area of cost for production of renewable materials is hydrolytic enzymes. Stephanopoulos, Challenges in Engineering Microbes for Biofuels Production, states “[d]espite substantial reduction in the cost of cellulolytic enzymes [ ], sugar release from biomass still remains an expensive and slow step, perhaps the most critical in the overall process.”

Himmel et al, Biomass recalcitrance: Engineering plants and enzymes for biofuels production, states the “cost-competitive production of biofuels is currently prevented by the high cost of biomass feedstocks and the processes for converting biomass to sugars—that is, the cost of the thermochemical pretreatment and enzyme hydrolysis unit operations in a biorefinery.”

Sticklen, Plant genetic engineering to improve biomass characteristics for biofuels, states the “idea that fermentable sugars for use in the production of alcohol fuels could be derived from crop biomass has been well received by the US Federal government; however, major economical downsides of biomass refineries include the pretreatment processing of the lignocellulosic matter and the cost of production of the microbial cellulases needed to convert the cellulose of biomass into fermentable sugars.”

Aden et al., Lignocellulosic Biomass to Ethanol Process Design and Economics Utilizing Co-current Dilute Acid Prehydrolysis and Enzymatic Hydrolysis for Corn Stover, published by the National Renewable Energy Laboratory discloses an ethanol plant process design using corn stover for a feedstock. The entire teachings of Aden et al. are hereby incorporated by reference in their entirety.

However, even with the above improvements in the processes, there is a need and a desire to reduce usage enzyme costs and produce renewable materials in a more cost effective manner.

### SUMMARY

This invention relates to an enzyme recovery sorbent, an enzyme recovery unit, a lignocellulosic biorefinery, a process for recycling enzymes, and a renewable material. This invention may reduce usage enzyme costs and produce renewable materials in a more cost effective manner. Hydrolytic enzymes act as a catalyst and are not consumed in reactions to depolymerize lignocellulosic material. Methods to recycle the enzymes can reduce operating costs and production costs of the renewable material.

According to a first embodiment, the invention includes a lytic enzyme recovery sorbent suitable for use in production of renewable materials. The sorbent includes a substrate, and an enzyme binding material dispersed with respect to the substrate.

According to a second embodiment, the invention includes a lytic enzyme recovery unit suitable for use in production of renewable materials. The unit includes a feed line for receiving a feed stream with lytic enzymes, and at least one enzyme recovery device with an enzyme sorbent for sorbing the lytic enzymes where the at least one enzyme recovery device is in fluid communication with the feed line. The unit also includes a regeneration line for supplying a release material to the at least one enzyme recovery device where the regeneration line is in fluid communication with the at least one enzyme recovery device. The unit also includes a recycle line for removing the lytic enzymes from the at least one enzyme recovery device where the recycle line is in fluid communication with the enzyme recovery device.

According to a third embodiment, the invention includes a lignocellulosic biorefinery suitable for production of renewable materials. The biorefinery includes a hydrolysis unit for depolymerization of cellulose and hemicellulose to a renewable-based feedstock by use of lytic enzymes, and a conversion unit for receiving at least a portion of the renewable-based feedstock and converting into a renewable material. The biorefinery also includes a separation unit for receiving a conversion unit effluent and forming a renewable material containing stream and a byproduct stream, and at least one enzyme recovery unit for receiving either the renewable material containing stream or the byproduct stream where the at least one enzyme recovery unit sorbs lytic enzymes for recycle. The biorefinery also includes a regeneration line to supply release material for release of the lytic enzymes from the at least one enzyme recovery unit, and a recycle line from the at least one enzyme recovery unit to the hydrolysis unit for recycling the lytic enzymes. The biorefinery also includes a product line from the at least one enzyme recovery unit or the conversion unit for the discharge of the renewable material, and a byproduct line from the at least one enzyme recovery unit or the separation unit for the discharge of the byproduct.

According to a fourth embodiment, the invention includes a process of recycling lytic enzymes suitable for production of renewable materials. The process includes the step of sorbing lytic enzymes on an enzyme sorbent to separate the lytic enzymes from a remainder of an input stream, and the step of releasing the lytic enzymes from the enzyme sorbent.

According to a fifth embodiment, the invention includes a renewable material made using the lytic enzyme recovery sorbent, the lytic enzyme recovery unit, the lignocellulosic biorefinery, and/or the process for recycling enzymes described herein.

### BRIEF DESCRIPTION OF THE DRAWINGS

The accompanying drawings, which are incorporated in and constitute a part of this specification, illustrate embodiments of the invention and, together with the description, serve to explain the features, advantages, and principles of the invention. In the drawings:

FIG. 1 illustrates enzymes and lignin in solution, according to one embodiment;

FIG. 2 illustrates enzymes bound to lignin in solution, according to one embodiment;

FIG. 3 illustrates enzymes and sorbent in solution, according to one embodiment;

FIG. 4 illustrates enzymes bound to sorbent, according to one embodiment;

FIG. 5 illustrates enzymes released from sorbent, according to one embodiment;

FIG. 6 illustrates an enzyme recovery unit, according to one embodiment;

FIG. 7 illustrates an enzyme recovery unit, according to one embodiment;

FIG. 8 illustrates a biorefinery, according to one embodiment;

FIG. 9 illustrates a biorefinery with a single enzyme recovery unit, according to one embodiment, and

FIG. 10 illustrates an ethanol plant, according to one embodiment.

### DETAILED DESCRIPTION

This invention may include an enzyme recovery sorbent, an enzyme recovery unit, a lignocellulosic biorefinery, a process for recycling enzymes, and/or a renewable material.

According to one embodiment, the invention may include applications of enzyme recovery from lignocellulose hydrolysis solutions. The application may include absorption of cellulase enzymes onto a solid-support-immobilized lignin for removal of the enzyme from a bulk hydrolysis solution. The application may also include suspension into an appropriately buffered and/or pH-adjusted solution, such as to disrupt the lignin-enzyme complex and release the cellulase enzyme.

Desirably, but not necessarily, the lignin-enzyme interaction can be dependent on protein confirmation resulting in selectivity for appropriately folded proteins. The recovery and recycle of cellulase enzymes can improve the economic viability of lignocellulosic conversion to renewable materials.

According to one embodiment, the invention may include methods for recovery and recycle of lytic enzymes. Solid-support immobilized lignin can be used to absorb lytic enzymes from a bulk hydrolysis solution, for example. The solid support with both embedded lignin and absorbed lytic enzymes can be suspended in an appropriately buffered and/or pH-adjusted aqueous solution. This solution can interrupt the lignin-enzyme interaction to release the enzyme for recycle back into the bulk hydrolysis solution. Recycle of lytic enzymes can reduce the total amount of lytic enzymes needed for lignocellulose hydrolysis and conversion to renewable materials, such as fuels and/or chemicals.

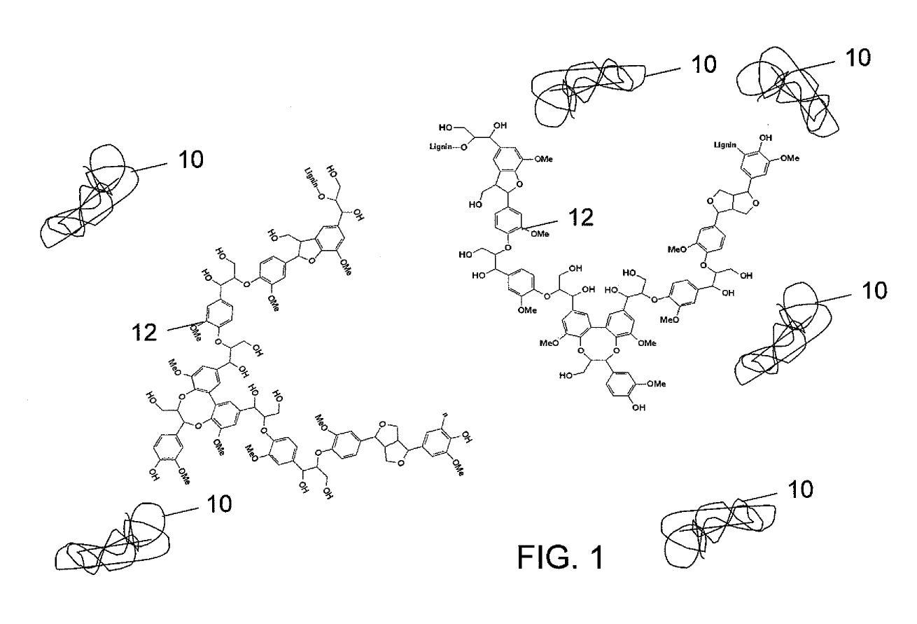

FIG. 1 illustrates enzymes 10 and lignin 12 in solution, according to one embodiment. The enzymes 10 may be folded and may be useful for hydrolysis of cellulose and/or hemicellulose. The lignin 12 may be from any suitable source, such as hardwoods, softwoods and/or the like. Suitable types of lignin 12 may include kraft lignin, klason lignin, acid-soluble lignin, acid-insoluble lignin, hardwood lignin, softwood lignin, agricultural material lignin, genetically-modified lignin, derivatized lignin, modified lignin, and/or the like. The lignin may have any suitable molecular weight, such as about 1700 grams per mole. Desirably, the lignin 12 may include any suitable number of sites or locations for receiving the enzymes 10, such as at least about one site, at least about two sites, at least about three sites, at least about five sites, at least about ten sites, and/or the like.

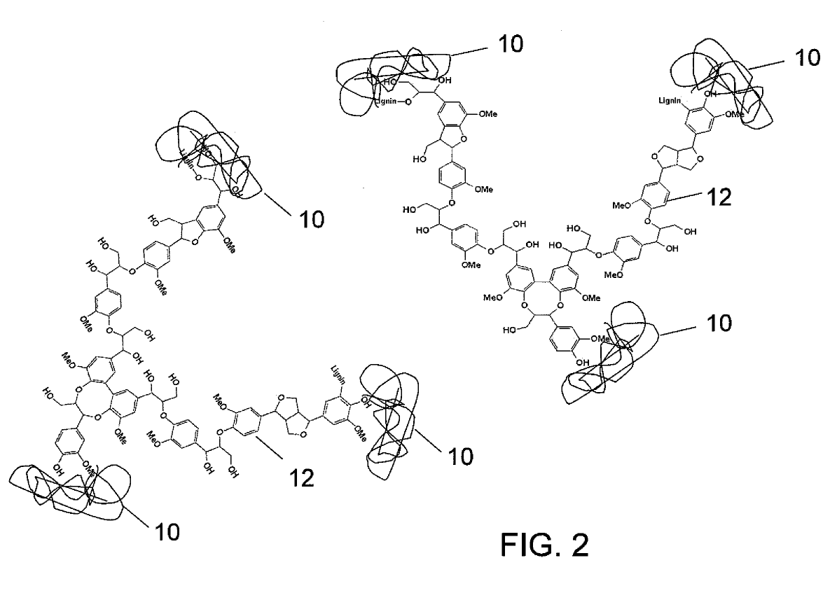

FIG. 2 illustrates enzymes 10 bound to lignin 12 in solution, according to one embodiment. The enzymes 10 can interact with a portion of the lignin 12 and become bound to each other, such as by strong and/or weak chemical bonds. Desirably, the lignin 12 and the enzymes 10 have a particular orientation and/or location for binding.

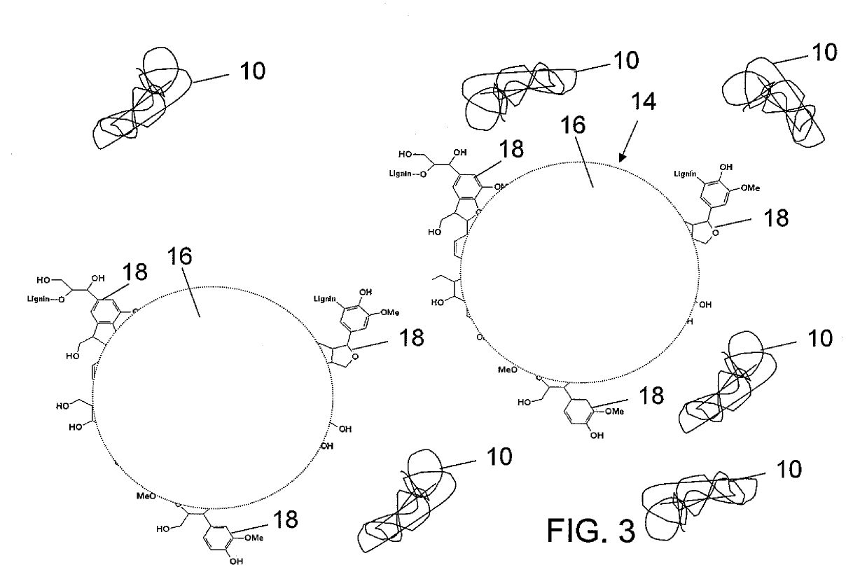

FIG. 3 illustrates enzymes 10 and sorbent 14 in solution, according to one embodiment. The sorbent 14 includes a substrate 16 with enzyme binding material 18 disposed on and/or impregnated into the substrate 16. Desirably, each particle of the sorbent 14 includes multiple locations for binding enzymes 10.

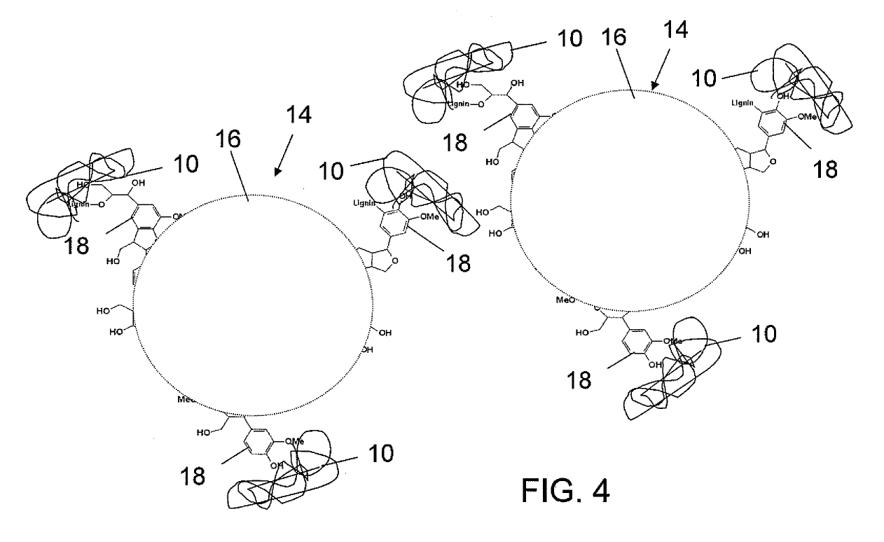

FIG. 4 illustrates enzymes 10 bound to sorbent 14, according to one embodiment. Desirably, but not necessarily the enzyme binding material 18 has at least a portion exposed with respect to the substrate 16.

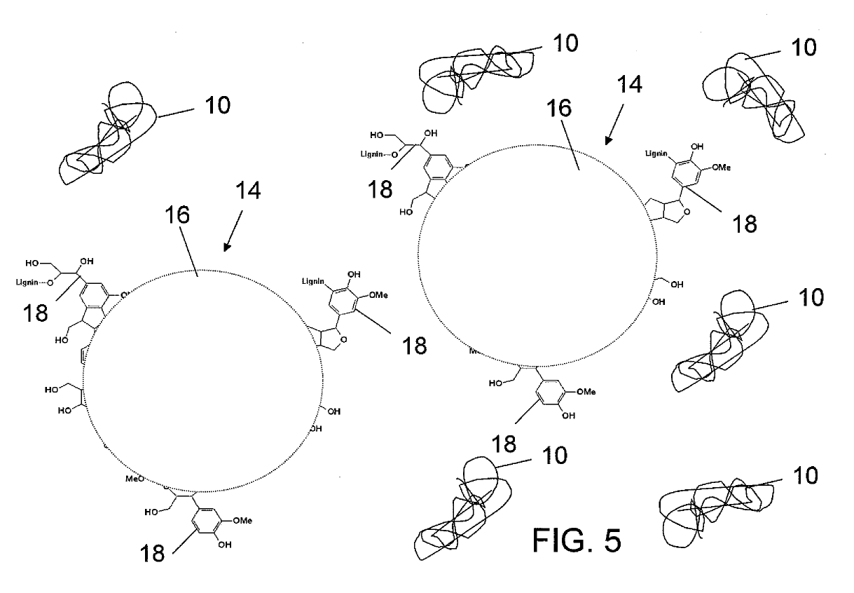

FIG. 5 illustrates enzymes 10 released from the sorbent 14, according to one embodiment. The releasing may occur by any suitable change, such as a change in pH, a change in osmolarity, a change in polarity, a change in temperature, and/or the like.

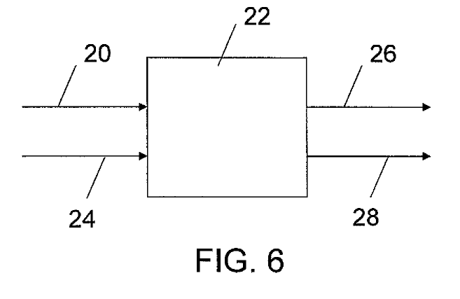

FIG. 6 illustrates an enzyme recovery unit 22, according to one embodiment. The enzyme recovery unit 22 includes a feed line 20 for supplying an enzyme containing material to the enzyme recovery unit 22. The enzyme recovery unit 22 also includes a regeneration line 24 for supplying a release material into the enzyme recovery unit 22. The enzyme recovery unit 22 also includes a recycle line 26 for delivering the enzymes 10 (not shown) that have been captured and released from the enzyme recovery unit 22. The enzyme recovery unit 22 also includes an outlet line 28 for delivering other materials contained in the feed from the enzyme recovery unit 22. The other materials may include renewable materials, saccharides, carbohydrates, biofuels, byproducts, and/or the like.

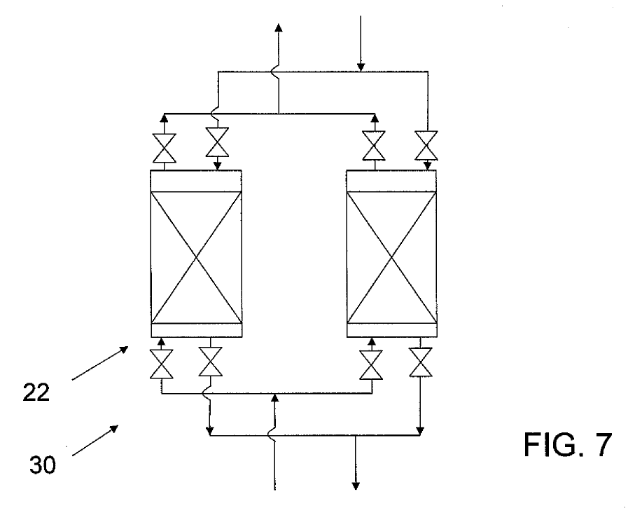

FIG. 7 illustrates an enzyme recovery unit 22, according to one embodiment. The enzyme recovery unit 22 includes two packed beds 30 and the associated piping and/or valves, such as for operation in series and/or parallel flow. The packed beds 30 may use any suitable arrangement of flow, such as up-flow sorption and up-flow regeneration, up-flow sorption and down-flow regeneration, down-flow sorption and down-flow regeneration, and/or the like.

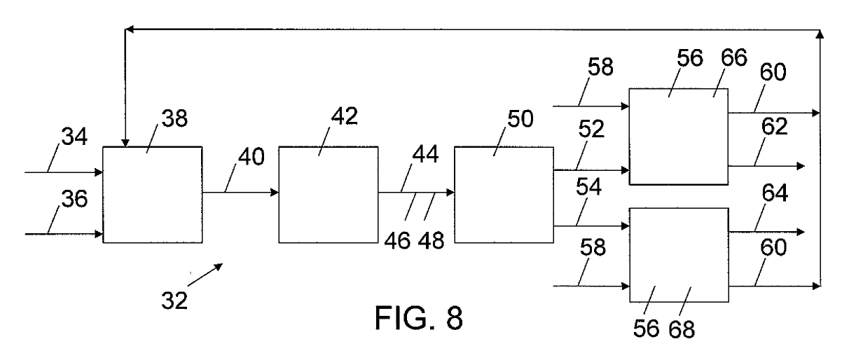

FIG. 8 illustrates a biorefinery 32, according to one embodiment. The biorefinery 32 includes a feedstock line 34 connected to the hydrolysis unit 38, such as for supplying lignocellulosic material. The biorefinery 32 also includes a fresh enzyme line 36 connected to the hydrolysis unit 38, such as for supplying make up enzymes. The hydrolysis unit 38 breaks down or depolymerizes the lignocellulosic material and may include any suitable pretreatment steps, processes, and/or devices. The pretreatment may include chemical, mechanical, and/or thermal processing including use of acids and/or bases, such as to convert hemicellulose into pentose or 5 carbon sugars. The hydrolysis unit 38 breaks down or depolymerizes the cellulose into hexose or 6 carbon sugars. Optionally and/or additionally, the hydrolysis unit 38 can also break down or depolymerize the hemicellulose into pentose or 5 carbon sugars, such was with hemicellulase.

The biorefinery 32 also includes a renewable-based feedstock line 40 connected to the hydrolysis unit 38 and a conversion unit 42, such as for flowing material containing hexose to the conversion unit 42. The conversion unit 42 converts the hexose into a renewable material 46 and a byproduct material 48 flowing in a conversion unit effluent line 44 connected to the conversion unit 42 and a separation unit 50. The renewable material 46 may be a biofuel and/or the like. The byproduct material 48 may include lignin. The conversion unit 50 may include one or more fermentors and/or one or more cell culture devices.

The separation unit 50 can use mechanical, thermal, and/or chemical, principles for separating or splitting the conversion unit effluent into a renewable material containing line 52 and a byproduct line 54. A centrifuge may be used in the separation unit 50. The renewable material containing, line 52, connects with the separation unit 50 and an enzyme recovery unit 56, such as a product enzyme recovery unit 66. The byproduct line 54 connects with the separation unit 50 and an enzyme recovery unit 56, such as a byproduct enzyme recovery unit 68.

A regeneration line 58 connects with each enzyme recovery unit 56, such as for supplying a release material to the enzyme recovery unit 56. A recycle line 60 connects with each enzyme recovery unit 56, such as for supplying the recovered or recycled enzymes to the hydrolysis unit 38.

A product line 62 connects with the product enzyme recovery unit 66, such as for flowing a renewable material to storage, for subsequent processing, for sale, and/or the like. A byproduct line 64 connects with the byproduct enzyme recovery unit 68, such as for flowing or moving a byproduct material to storage, disposal, and/or the like. Configurations of the biorefinery 32 with one enzyme recovery unit 56 are within the scope of this invention (only on the product line or only on the byproduct line).

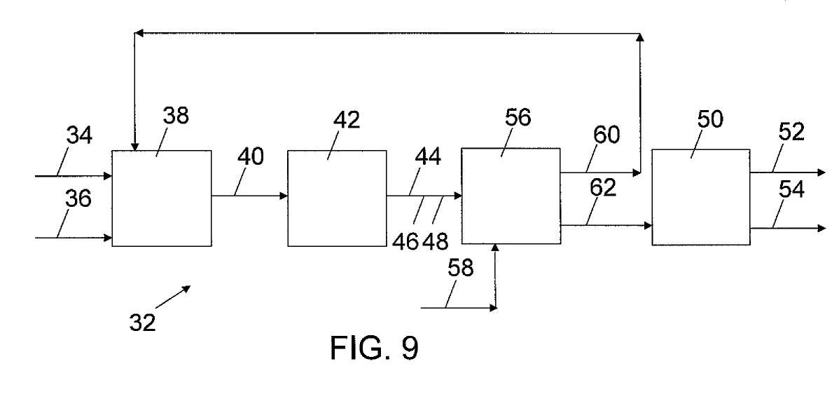

FIG. 9 illustrates a biorefinery 32 with a single enzyme recovery unit 56, according to one embodiment. The biorefinery 32 includes a feedstock line 34 connected to the hydrolysis unit 38, such as for supplying lignocellulosic material. The biorefinery 32 also includes a fresh enzyme line 36 connected to the hydrolysis unit 38, such as for supplying make up enzymes.

The biorefinery 32 also includes a renewable-based feedstock line 40 connected to the hydrolysis unit 38 and a conversion unit 42, such as for flowing material containing hexose to the conversion unit 42. The conversion unit 42 converts the hexose into a renewable material 46 and a byproduct material 48 both flowing in a conversion unit effluent line 44 connected to the enzyme recovery unit 56.

A regeneration line 58 connects with the enzyme recovery unit 56, such as for supplying a release material to the enzyme recovery unit 56. A recycle line 60 connects with the enzyme recovery unit 56, such as for supplying the recovered or recycled enzymes to the hydrolysis unit 38.

A product line 62 connects with the enzyme recovery unit 56, such as for flowing the renewable material and the byproduct to a separation unit 50. The separation unit 50 separates or splits the enzyme recovery unit effluent into a renewable material containing line 52 and a byproduct line 54. Other configurations of the biorefinery 32 are within the scope of this invention.

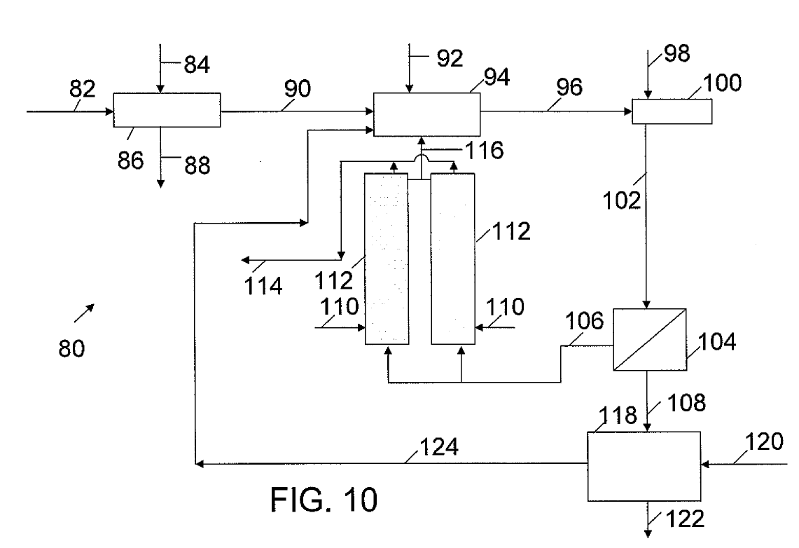

FIG. 10 illustrates an ethanol plant 80, according to one embodiment. The ethanol plant 80 uses a lignocellulose line 82 to receive feed material to a pretreatment unit 86, such as for depolymerizing hemicellulose with an acid, base, steam, and/or the like. The pretreatment unit 86 receives catalyst by a catalyst line 84 and A pentose line 88 flows a sugar solution from the pretreatment unit 86, such as for subsequent processing or conversion. The pretreatment unit 86 connects to a hydrolysis unit 94 by a cellulose line 90, such as for flowing cellulose and lignin.

The hydrolysis unit 94 receives fresh enzymes from an enzyme line 92 and recycle enzymes from recovery enzyme lines 116 and 124. Hexose, lignin, and enzymes flow in a hexose line 96 from the hydrolysis unit 94 and connects to the fermentation unit 100.

The fermentation unit 100 receives organisms by an organism line 98. Organisms may include bacteria, fungi, algae, and/or the like. The fermentation unit 100 converts the hexose into ethanol. Ethanol, lignin, and enzymes flow in a fermentation effluent line 102 connected to the fermentation unit 100 and a separation unit 104.

The separation unit 104 separates ethanol and water from the solid lignin using ethanol line 106 and lignin line 108. Enzymes from the separation unit 104 flow with both the ethanol and the lignin. The ethanol line 106 connects the separation unit 104 with enzyme recovery columns 112, such as in a primary enzyme recovery unit. The lignin line 108 connects the separation unit 104 with a secondary enzyme recovery unit 118. Recovery buffer lines 110 and 120 supply release material to the enzyme recovery columns 112 and the secondary enzyme recovery unit 118.

A product ethanol line 114 flows ethanol and water from the enzyme recovery columns 112, such as for product recovery, distillation, storage, and/or the like. A lignin line 122 flows or moves the solid lignin, such as with a conveyor, auger, gravity, and/or the like. The dual column configuration of FIG. 10 can provide continuous operations while providing for regeneration of a spent bed full of enzymes.

Renewable material broadly refers to a substance or item that has been at least partially derived from a source or process capable of being replaced by natural ecological cycles or resources. Renewable materials may broadly include chemicals, chemical intermediates, solvents, monomers, oligomers, polymers, biofuels, biofuel intermediates, biogasoline, biogasoline blendstocks, biodiesel, green diesel, renewable diesel, biodiesel blend stocks, biodistillates, and/or the like. Desirably, but not necessarily, the renewable material may be derived from a living organism, such as plants, algae, bacteria, fungi, and/or the like.

Biofuel broadly refers to components or streams suitable for use as a fuel or a combustion source derived from renewable sources, such as may be sustainably be produced and/or have reduced or no net carbon emissions to the atmosphere. Renewable resources may exclude materials mined or drilled, such as from the underground. Desirably, renewable resources may include single cell organisms, multicell organisms, plants, fungi, bacteria, algae, cultivated crops, non-cultivated crops, timber, and/or the like.

Biogasoline broadly refers to components or streams suitable for direct use and/or blending into the gasoline or octane pool or supply derived from renewable sources, such as methane, hydrogen, syn (synthesis) gas, methanol, ethanol, propanol, butanol, dimethyl ether, methyl tert-buyl ether, ethyl tert-butyl ether, hexanol, aliphatic compounds (straight, branched, and/or cyclic), heptane, isooctane, cyclopentane, aromatic compounds, ethyl benzene, and/or the like. Butanol broadly refers to products and derivatives of 1-butanol, 2-butanol, iso-butanol, other isomers, and/or the like. Biogasoline may be used in spark ignition engines, such as automobile gasoline internal combustion engines. According to one embodiment, the biogasoline and/or biogasoline blends meet or comply with industrially accepted fuel standards.

Biodiesel broadly refers to components or streams suitable for direct use and/or blending into the diesel or cetane pool or supply derived from renewable sources, such as fatty acid esters, triglycerides, lipids, fatty alcohols, alkanes, naphthas, distillate range materials, paraffinic materials, aromatic materials, aliphatic compounds (straight, branched, and/or cyclic), and/or the like. Biodiesel may be used in compression engines, such as automotive diesel internal combustion engines. In the alternative, the biodiesel may also be used in gas turbines, heaters, boilers, and/or the like. According to one embodiment, the biodiesel and/or biodiesel blends meet or comply with industrially accepted fuel standards.

Biodistillate broadly refers to components or streams suitable for direct use and/or blending into aviation fuels (jet), lubricant base stocks, kerosene fuels, and/or the like derived from renewable sources, and having a boiling point range of between about 100 degrees Celsius and about 700 degrees Celsius, between about 150 degrees Celsius and about 350 degrees Celsius, and/or the like.

According to one embodiment, the invention may include a lytic enzyme recovery sorbent suitable for use in production of renewable materials. The sorbent may include a substrate, and an enzyme binding material dispersed with respect to the substrate.

Lytic broadly refers to or relates to a process or step of breaking down, disintegration, dissolution, lysis, and/or the like.

Enzymes broadly refer to proteins or other suitable molecules to catalyze and/or increase chemical reactions, biochemical reactions, and/or the like. Enzymes may be produced by living organisms and/or synthetic processes. Suitable enzymes may include any desirable property. Suitable enzymes may include cellulase, hemicellulase, ligninase, endo-cellulase, exo-cellulase, glucosidase, cellobiose dehydrogenase, manganese peroxidase, lignin peroxidase, and/or the like, such as to aid hydrolysis of cellulose to smaller sugar units and/or monomers.

Recovery broadly refers the act, process, and/or instance of taking back and/or preparing for reuse, recycle, separation, and/or the like.

Sorbent broadly refers to a substance that can take up and hold an atom or a molecule, such as by absorption, adsorption, chemisorption, physisorption, ion exchange, and/or the like.

Absorption broadly refers to a physical process and/or a chemical process in which atoms, molecules, and/or ions enter a bulk phase, such as into a gas, a liquid, a solid material, and/or the like.

Adsorption broadly refers to a process that occurs when a gas solute and/or a liquid solute accumulates on the surface of a solid adsorbent and/or a liquid adsorbent, such as to form a film of molecules or atoms as an adsorbate, and/or the like.

Chemisorption broadly refers to a classification of adsorption characterized by a strong interaction between an adsorbate and a surface, and/or the like.

Physisorption broadly refers to a classification of adsorption characterized by a weak Van der Waals force between an adsorbate and a surface, and/or the like.

Ion exchange broadly refers to is an exchange or replacement of ions between two electrolytes, between an electrolyte solution and a complex or a molecule, and/or the like.

Substrate broadly refers to an underlying support and/or foundation, such as a substance with a permanent subject of qualities and/or the like. Desirably, the substrate for the sorbent is at least generally inert with respect to the processes or environments in which it can be used. Also desirably, the substrate may include an at least generally high surface area relative to a volume of the substrate. The substrate may include any suitable material and/or composition.

Particles or pieces of substrate may include any suitable size and/or shape. Porous substrates may include a relatively larger size and/or a block shape. In the alternative, the particles or pieces of substrate may include an at least generally spherical shape. The size or effective diameter of the substrate particle may be a function of diffusion processes and/or the like. The substrate particle may have an effective diameter of between about 1 micrometer to about 10 centimeters, between about 1 millimeter to about 2 centimeters, and/or the like.

Enzyme binding material broadly refers to any suitable substance that can interact with an enzyme or other suitable molecule to capture, grab, hold, and/or the like the enzyme. The binding interaction may include any suitable forces, such as ionic bonding, covalent bonding, hydrogen bonding, Vander Waal forces, strong molecular forces, weak molecular forces, physical forces, mechanical forces, and/or the like.

Dispersed broadly refers to being distributed or spread widely and/or disseminated. Desirably, a state of being dispersed includes distributed at least relatively evenly on a surface of and/or impregnated throughout a medium or a material, such as the substrate. According to one embodiment, the enzyme binding material may be the substrate itself, such as without other or additional support media.

According to one embodiment, the substrate may include alginate, agar, polyacrylamide, collagen, activated carbon, porous ceramic, diatomaceous earth, nylon, cellulose, polysulfone, polyacrylate, alumina, silica, bentonite, ion exchange resin, and/or the like.

According to one embodiment, the enzyme binding material may include lignin and/or the like. Desirably, but not necessarily, lignin used as a binding material may be different than lignin in a feedstock material, such as the lytic enzymes had a low binding efficiency with respect to the lignin in the feedstock material and a higher binding efficiency with respect to the lignin of the binding material. A ratio of the binding efficiency of the enzymes to the binding material to the binding efficiency of the enzymes to the feedstock material may be at least about 2:1, at least about 5:1, at least about 10:1, at least about 50:1, and/or the like.

In the alternative, the enzyme binding material may include macro-molecular substructures of lignin, such as para-coumaryl alcohol, coniferyl alcohol, sinapyl alcohol, and/or the like. Combinations of lignin and macro-molecular substructures of lignin for the enzyme binding material are within the scope of the invention.

According to one embodiment, the enzyme binding material can have an average molecular weight of between about 300 atomic mass units to about 1,000,000 atomic mass units, between about 1,000 atomic mass units to about 100,000 atomic mass units, between about 5,000 atomic mass units to about 50,000 atomic mass units, and/or the like.

The enzyme binding material may include any suitable enzyme binding efficiency on a molar basis and/or other suitable basis. Enzyme binding efficiency broadly refers to an amount of the enzyme captured and/or retained by the enzyme binding material, such as when the enzyme contacts the enzyme binding material. According to one embodiment, the enzyme binding material has a binding efficiency of at least about 60 percent on a molar basis, at least about 70 percent on a molar basis, at least about 80 percent on a molar basis, at least about 90 percent on a molar basis, at least about 95 percent on a molar basis, and/or the like.

Desirably, but not necessarily, the enzyme binding material reversibly sorbs the lytic enzyme, such as to release the enzyme upon a change in conditions and/or operation. According to one embodiment, the act or step of reversibly sorbing the lytic enzyme results in or yields of at least about 70 percent of the bound enzyme for reuse or processing, at least about 80 percent of the bound enzyme for reuse or processing, at least about 90 percent of the bound enzyme for reuse or processing, at least about 95 percent of the bound enzyme for reuse or processing and/or the like. Some loss or degradation of the lytic enzymes may occur.

Any suitable step or action may release or free the lytic enzyme from the enzyme binding material, such as upon application of a pH change, a temperature change, a buffer solution change, a solvent polarity change, and/or the like. A pH change may include going from neutral conditions to acidic conditions and/or basic conditions with a pH adjusting agent (acid or base), for example. A temperature change may include raising the temperature by about 10 degrees Celsius, for example. A buffer solution change may include adding a different salt to the solution, for example. A solvent polarity change may include changing from an aqueous solution to an alcohol solution, for example. Desirably, the step to release the enzyme seeks to minimize degradation and/or loss of the enzyme.

According to one embodiment, the invention may include a renewable material made with or by the sorbent described above.

According to one embodiment, the invention may include a lytic enzyme recovery unit suitable for use in production of renewable materials. The unit may include a feed line for receiving a feed stream with lytic enzymes, and at least one enzyme recovery device with an enzyme sorbent for sorbing the lytic enzymes. The at least one enzyme recovery device can be in fluid communication with the feed line. The unit may also include a regeneration line for supplying a release material to the at least one enzyme recovery device. The regeneration line can be in fluid communication with the at least one enzyme recovery device. The unit may also include a recycle line for removing the lytic enzymes from the at least one enzyme recovery device. The recycle line can be in fluid communication with the enzyme recovery device.

Unit broadly refers to one or more pieces of equipment or devices used in a process or a task. The enzyme recovery unit can remove and/or recover enzymes from a stream and then provide or supply them for reuse, for example.

Line broadly refers to a device for connecting and/or transmitting a substance or a material, such as a pipe, a channel, a conduit, a path, and/or the like. The feed line can provide an inlet into the unit with the lytic enzymes to be recycled, for example. The regeneration line can provide a release material to allow the enzyme to be loosed for recycle, for example. The recycle line can provide an outlet from the unit for the lytic enzymes, for example.

Stream broadly refers to a flow or a passage of a material or a substance.

Enzyme recovery device broadly refers to any suitable process equipment or device for capturing the enzymes from the stream. The enzyme recovery device may include a sorbent material, such as a packed bed, a packed column, a fluidized bed, and/or the like. The enzyme recovery device may include multiple vessels, such as one or more in sorption (on-line) mode, one or more in stand by (ready) mode, one or more in regeneration mode (off-line), and/or the like.

According to one embodiment, the at least one enzyme recovery device comprises two or more packed beds each operable in sorption mode and regeneration mode.

The sorbent used in the at least one enzyme recovery device may include any suitable material. According to one embodiment, the enzyme sorbent may include an enzyme binding material of lignin or macro-molecular substructures of lignin including para-coumaryl alcohol, coniferyl alcohol, sinapyl alcohol, and/or the like. The enzyme sorbent may also include a substrate for supporting the enzyme binding material of alginate, agar, polyacrylamide, collagen, activated carbon, porous ceramic, diatomaceous earth, nylon, cellulose, polysulfone, polyacrylate, alumina, silica, bentonite, ion exchange resin, and/or the like. The enzyme binding material can have an average molecular weight of between about 300 atomic mass units to about 1,000,000 atomic mass units, for example.

According to one embodiment, the enzyme sorbent used in the at least one enzyme recovery device can have a reversible binding efficiency of at least about 60 percent on a molar basis.

The release material from the regeneration line may provide any suitable action or step to free the captured enzymes, such as a pH change, a temperature change, a buffer solution change, a solvent polarity change, and/or the like.

According to one embodiment, the feed stream may also include a renewable-based material or a byproduct material, such as from a fermentation unit and/or the like. The renewable-based material in the feed stream may include any suitable substance, such as ethanol, butanol, free fatty acids, triacylglycerides, alkyl esters, isoprenoids, lactic acid, acetic acid, butyric acid, propionic acid, any of the biofuels described above, and/or the like.

The feed stream may also include a lignocellulosic hydrolyzate material, such as with carbohydrates, sugar monomers, sugar dimers, sugar trimers, sugar oligomers, and/or the like, according to one embodiment

According to one embodiment, the invention may include a renewable material made in or by the lytic enzyme recovery unit described above.

According to one embodiment, the invention may include a lignocellulosic biorefinery suitable for production of renewable materials. The biorefinery may include a hydrolysis unit for depolymerization of cellulose and/or hemicellulose to a renewable-based feedstock by use of lytic enzymes, and a conversion unit for receiving at least a portion of the renewable-based feedstock and converting into a renewable material. The biorefinery may also include a separation unit for receiving a conversion unit effluent and forming a renewable material containing stream and a byproduct stream, and at least one enzyme recovery unit for receiving the conversion unit effluent, the renewable material containing stream or the byproduct stream. The at least one enzyme recovery unit can sorb lytic enzymes for recycle. The biorefinery may also include a regeneration line to supply release material for release of the lytic enzymes from the at least one enzyme recovery unit, and a recycle line from the at least one enzyme recovery unit to the hydrolysis unit for recycling the lytic enzymes. The biorefinery may also include a product line from the at least one enzyme recovery unit or the conversion unit for the discharge of the renewable material, and a byproduct line from the at least one enzyme recovery unit or the separation unit for the discharge of the byproduct.

Biorefinery broadly refers to the plant or collection of units used to produce a renewable material.

Lignoellulosic broadly refers to containing cellulose, hemicellulose, lignin, and/or the like, such as plant material. Lignocellulosic material may include any suitable material, such as sugar cane, sugar cane bagasse, energy cane bagasse, rice, rice straw, corn, corn stover, wheat, wheat straw, maize, maize stover, sorghum, sorghum stover, sweet sorghum, sweet sorghum stover, cotton remnant, sugar beet, sugar beet pulp, soybean, rapeseed, jatropha, switchgrass, miscanthus, other grasses, timber, softwood, hardwood, wood waste, sawdust, paper, paper waste, agricultural waste, municipal waste, any other suitable biomass material, and/or the like.

The units and/or lines of the biorefinery may include any and/or all of the characteristics and/or features described above.

The hydrolysis unit may include any suitable step, equipment, and/or process for depolymerizing of lignin, cellulose, and/or hemicellulose to a renewable-based feedstock by use of lytic enzymes. Desirably, the renewable-based feedstock may include six-carbon and five-carbon sugars (hexose and pentose, respectively), glucose, xylose, and/or other materials that can be converted to a renewable material. The hydrolysis unit may also include any suitable pretreatment processes or equipment for chemical, thermal, and/or mechanical systems, such as steam contacting (temperature), acid contacting, base contacting, pulverizing, shredding, and/or the like. The hydrolysis unit may include a vessel, a reactor, a tank, a basin, an agitator, a bubbler and/or the like. The hydrolysis unit may receive recovered or recycle enzymes from a recycle enzyme line. Fresh or new enzymes may also be supplied to the hydrolysis unit.

Depolymerizing broadly refers to taking something larger and breaking it into smaller units or pieces. Depolymerizing may include breaking or severing chemical bonds, such as to release monomers (1 unit) from a polymeric backbone or chain. Depolymerizing may also produce dimers (2 units), trimers (3 units), tetramers (4 units), any other suitable oligomers (few units), and/or the like, such as intermediates and/or complete products.

Lignin broadly refers to a biopolymer that may be part of secondary cell walls in plants, such as a complex highly cross-linked aromatic polymer that may covalently link to hemicellulose.

Hemicellulose broadly refers to a branched sugar polymer composed mostly of pentoses, such as with a generally random amorphous structure and typically may include up to hundreds of thousands of pentose units.

Cellulose broadly refers to an organic compound with the formula (C₆H₁₀O₅)_{z} where z includes any suitable integer. Cellulose may include a polysaccharide with a linear chain of several hundred to over ten thousand hexose units and a high degree of crystalline structure, for example.

The conversion unit may include any suitable step, equipment, and/or process for receiving at least a portion of the renewable-based feedstock and converting into a renewable material. According to one embodiment, the conversion unit utilizes fermentation processes and/or cell culture processes.

Fermentation broadly refers to the metabolism of carbohydrates whereby the final electron donor is not oxygen, such as anaerobically. Fermentation may include an enzyme controlled anaerobic breakdown of an energy-rich compound, such as a carbohydrate to carbon dioxide and an alcohol and/or an organic acid. In the alternative, fermentation broadly refers to biologically controlled transformation of an organic compound. Fermentation processes may use any suitable organisms, such as bacteria, fungi, algae, and/or the like. Suitable fermentation processes may include naturally occurring organisms and/or genetically modified organisms.

Cell culturing broadly refers to the metabolism of carbohydrates whereby the final electron donor is oxygen, such as aerobically. Cell culturing processes may use any suitable organisms, such as bacteria, fungi, algae, and/or the like. Suitable cell culturing processes may include naturally occurring organisms and/or genetically modified organisms.

The separation unit may include any suitable step, equipment, and/or process for receiving a conversion unit effluent and forming a renewable material containing stream and a byproduct stream. The separation unit may include one of more distillation columns, packed beds, mechanical devices, centrifuges, and/or the like.

According to one embodiment, the at least one enzyme recovery unit may include a product enzyme recovery unit and a byproduct enzyme recovery unit. The product enzyme recovery unit and the byproduct enzyme recovery unit may share or combine common pieces of equipment and/or processes. The product enzyme recovery unit may recover enzymes from the renewable product stream, such as a biofuel. The byproduct enzyme recovery unit may recovery enzymes from the byproduct stream, such as lignin.

The biorefinery may use any suitable amount of recycled enzymes on any suitable basis, such as where at least about 35 percent of lytic enzymes supplied to the hydrolysis unit comprise recycled enzymes on a mass basis, at least about 45 percent of lytic enzymes supplied to the hydrolysis unit comprise recycled enzymes on a mass basis, at least about 55 percent of lytic enzymes supplied to the hydrolysis unit comprise recycled enzymes on a mass basis, at least about 65 percent of lytic enzymes supplied to the hydrolysis unit comprise recycled enzymes on a mass basis, at least about 75 percent of lytic enzymes supplied to the hydrolysis unit comprise recycled enzymes on a mass basis, at least about 85 percent of lytic enzymes supplied to the hydrolysis unit comprise recycled enzymes on a mass basis, at least about 95 percent of lytic enzymes supplied to the hydrolysis unit comprise recycled enzymes on a mass basis, and/or the like.

According to one embodiment, the biorefinery uses a lignocellulosic feedstock to produce ethanol, butanol, free fatty acids, triacylglycerides, alkyl esters, isoprenoids, lactic acid, acetic acid, butyric acid, propionic acid, and/or the like.

According to one embodiment, the invention may include a renewable material made in the or by the biorefinery described above.

According to one embodiment, the invention may include a process of recycling lytic enzymes suitable for production of renewable materials. The process may include the step of sorbing lytic enzymes on an enzyme sorbent to separate the lytic enzymes from a remainder of an input stream, and the step of releasing the lytic enzymes from the enzyme sorbent.

The step of sorbing lytic enzymes may include any suitable efficiency, such as at least about 60 percent on a molar basis and/or the like.

The step of releasing the lytic enzymes may include any suitable change, such as by a pH change, a temperature change, a buffer solution change, a solvent polarity change, and/or the like.

The process may also include the step of adding lytic enzymes to hydrolyze cellulose or hemicellulose to a convertible material and the step of converting the convertible material to a renewable material. The process may also include the step of separating byproduct from the renewable material, and the step of returning the released enzymes to be used for additional hydrolysis.

According to one embodiment, the invention may include a renewable material made in or by the process described above.

The scope of the invention is not limited merely to lytic enzymes, but broadly may be applicable to other enzymes or molecules. Similarly, the scope of the invention is not limited merely to production of renewable materials, but broadly may be applied or used with other processes and/or applications.

It will be apparent to those skilled in the art that various modifications and variations can be made in the disclosed structures and methods without departing from the scope or spirit of the invention. Particularly, descriptions of any one embodiment can be freely combined with descriptions or other embodiments to result in combinations and/or variations of two or more elements or limitations. Other embodiments of the invention will be apparent to those skilled in the art from consideration of the specification and practice of the invention disclosed herein. It is intended that the specification be considered exemplary only, with a true scope and spirit of the invention being indicated by the following claims.

What is claimed is:

1. A lytic enzyme recovery sorbent suitable for use in production of renewable materials, the sorbent comprising:
a substrate; and
an enzyme binding material dispersed with respect to the substrate.

2. The sorbent of claim 1, wherein the substrate comprises alginate, agar, polyacrylamide, collagen, activated carbon, porous ceramic, diatomaceous earth, nylon, cellulose, polysulfone, polyacrylate, alumina, silica, bentonite, or ion exchange resin.

3. The sorbent of claim 1, wherein the enzyme binding material comprises lignin or macro-molecular substructures of lignin comprising para-coumaryl alcohol, coniferyl alcohol, or sinapyl alcohol.

4. The sorbent of claim 1, wherein the enzyme binding material has an average molecular weight of between about 300 atomic mass units to about 1,000,000 atomic mass units.

5. The sorbent of claim 1, wherein the enzyme binding material has a binding efficiency of at least about 60 percent on a molar basis.

6. The sorbent of claim 1, wherein the enzyme binding material reversibly sorbs the lytic enzyme.

7. The sorbent of claim 1, wherein the enzyme binding material releases the lytic enzyme upon application of a pH change, a temperature change, a buffer solution change, or a solvent polarity change.

8. A renewable material made using the sorbent of claim 1.

9. A lytic enzyme recovery unit suitable for use in production of renewable materials, the unit comprising:
a feed line for receiving a feed stream comprising lytic enzymes;
at least one enzyme recovery device comprising an enzyme sorbent for sorbing the lytic enzymes, the at least one enzyme recovery device in fluid communication with the feed line;
a regeneration line for supplying a release material to the at least one enzyme recovery device, the regeneration line in fluid communication with the at least one enzyme recovery device; and
a recycle line for removing the lytic enzymes from the at least one enzyme recovery device, the recycle line in fluid communication with the enzyme recovery device.

10. The unit of claim 9, wherein the at least one enzyme recovery device comprises two or more packed beds each operable in sorption mode and regeneration mode.

11. The unit of claim 9, wherein the enzyme sorbent comprises.
an enzyme binding material comprising lignin or macro-molecular substructures of lignin comprising para-coumaryl alcohol, coniferyl alcohol, or sinapyl alcohol; and
a substrate for supporting the enzyme binding material, the substrate comprising alginate, agar, polyacrylamide, collagen, activated carbon, porous ceramic, diatomaceous earth, nylon, cellulose, polysulfone, polyacrylate, alumina, silica, bentonite, or ion exchange resin;
wherein the enzyme binding material has an average molecular weight of between about 300 atomic mass units to about 1,000,000 atomic mass units.

12. The unit of claim 9, wherein the enzyme sorbent has a reversible binding efficiency of at least about 60 percent on a molar basis.

13. The unit of claim 9, wherein the release material provides a pH change, a temperature change, a buffer solution change, or a solvent polarity change.

14. The unit of claim 9, wherein feed stream further comprises a hydrolyzate material, a renewable-based material, or a byproduct material.

15. The unit of claim 14, wherein the renewable-based material comprises ethanol, butanol, free fatty acids, triacylglycerides, alkyl esters, isoprenoids, lactic acid, acetic acid, butyric acid, or propionic acid.

16. A renewable material made in the unit of claim 9.

17. A lignocellulosic biorefinery suitable for production of renewable materials, the biorefinery comprising:
a hydrolysis unit for depolymerization of cellulose and hemicellulose to a renewable-based feedstock by use of lytic enzymes;
a conversion unit for receiving at least a portion of the renewable-based feedstock and converting into a renewable material;
a separation unit for receiving a conversion unit effluent and forming a renewable material containing stream and a byproduct stream;
at least one enzyme recovery unit for receiving the conversion unit effluent, the renewable containing stream, or the byproduct stream, the at least one enzyme recovery unit sorbing lytic enzymes for recycle;
a regeneration line to supply release material for release of the lytic enzymes from the at least one enzyme recovery unit;
a recycle line from the at least one enzyme recovery unit to the hydrolysis unit for recycling the lytic enzymes;
a product line from the at least one enzyme recovery unit or the conversion unit for the discharge of the renewable material; and
a byproduct line from the at least one enzyme recovery unit or the separation unit for the discharge of the byproduct.

18. The biorefinery of claim 17, wherein the at least one enzyme recovery unit comprises a product enzyme recovery unit and a byproduct enzyme recovery unit.

19. The biorefinery of claim 17, wherein at least about 35 percent of lytic enzymes supplied to the hydrolysis unit comprise recycled enzymes.

20. The biorefinery of claim 17, wherein the biorefinery uses a lignocellulosic feedstock to produce ethanol, butanol, free fatty acids, triacylglycerides, alkyl esters, isoprenoids, lactic acid, acetic acid, butyric acid, or propionic acid.

21. A renewable material made in the biorefinery of claim 17.

22. A process of recycling lytic enzymes suitable for production of renewable materials, the process comprising:
sorbing lytic enzymes on an enzyme sorbent to separate the lytic enzymes from a remainder of an input stream; and
releasing the lytic enzymes from the enzyme sorbent.

23. The process of claim 22, wherein the step of sorbing lytic enzymes has a binding efficiency of at least about 60 percent on a molar basis.

24. The process of claim 22, wherein the step of releasing the lytic enzymes occurs by a pH change, a temperature change, a buffer solution change, or a solvent polarity change.

25. The process of claim 22, further comprising:
adding lytic enzymes to hydrolyze cellulose or hemicellulose to a convertible material;
converting the convertible material to a renewable material;
separating byproduct from the renewable material; and
returning the released enzymes to be used for additional hydrolysis.

26. A renewable material made by the process of claim 22.
